(19) United States
(12) Patent Application Publication
(10) Pub. No.: US 2008/0160064 A1
(43) Pub. Date: Jul. 3, 2008

(54) DEVICES AND METHODS FOR INHIBITING FIBROSIS

(76) Inventors: Christopher C. Capelli, Houston, TX (US); Andrew P. Dennis, Houston, TX (US)

Correspondence Address: FULBRIGHT & JAWORSKI L.L.P., 600 CONGRESS AVE., SUITE 2400, AUSTIN, TX, 78701, US

(21) Appl. No.: 11/963,032

(22) Filed: Dec. 21, 2007

Related U.S. Application Data
(60) Provisional application No. 60/871,638, filed on Dec. 22, 2006.

(51) Int. Cl. A61F 2/02; A61K 33/00; A61P 17/00

(52) U.S. Cl. 424/423; 424/699

(57) ABSTRACT

The present invention relates to compositions and methods for rendering medical devices less prone to fibrous encapsulation comprising the use of non-gaseous CO dissolved in a reservoir material. Upon exposure to a biological environment, non-gaseous CO is slowly desorbed and/or dissolved from the reservoir material which provides anti-fibrotic activity. These compositions and methods may be used directly on soft tissue implants and/or medical devices to treat and/or prevent conditions and disease states related to fibrosis. Furthermore, the compositions can be incorporated into materials that are used to create soft tissue implants and/or medical devices to imbue implants and devices with anti-fibrotic properties.

### CROSS-REFERENCE TO RELATED APPLICATIONS

This application claims the priority of U.S. Provisional Patent Application Ser. No. 60/871,638, filed Dec. 22, 2006, the entire disclosure of which is specifically incorporated herein by reference.

### BACKGROUND OF THE INVENTION

I. Field of the Invention

The present invention relates to compositions containing non-gaseous carbon monoxide that may be used directly or incorporated into carriers to treat conditions and disease states arising from fibrotic side effects caused by implanted devices. The compositions can further be incorporated into or coat soft tissue implants and/or medical devices to provide anti-fibrotic surfaces.

II. Related Art

The use of soft tissue implants for cosmetic applications (aesthetic and reconstructive) is common in breast augmentation, breast reconstruction after cancer surgery, craniofacial procedures, reconstruction after trauma, congenital craniofacial reconstruction and oculoplastic surgical procedures to name a few.

The clinical function of a soft tissue implant depends upon the implant being able to effectively maintain its shape over time. In many instances, when these devices are implanted in the body, they are subject to a “foreign body” response from the surrounding host tissues. The body recognizes the implanted device as foreign, which triggers a cytokine response followed by encapsulation of the implant with fibrous connective tissue (adhesion formation). Encapsulation of surgical implants complicates a variety of reconstructive and cosmetic surgeries, but is particularly problematic in the case of breast reconstruction surgery where the breast implant becomes surrounded by a fibrous capsule that alters anatomy and function. Scar capsules that harden and contract (known as “capsular contractures”) are the most common complication of breast implant or reconstructive surgery. Capsular (fibrous) contractures can result in hardening of the breast, loss of the normal anatomy and contour of the breast, discomfort, weakening and rupture of the implant shell, asymmetry, infection, and patient dissatisfaction. Further, fibrous encapsulation of any soft tissue implant can occur even after a successful implantation if the device is manipulated or irritated by the daily activities of the patient. Bleeding in and around the implant can also trigger a biological cascade that ultimately leads to excess scar tissue formation. The effects of unwanted scarring in the vicinity of the implant are the leading cause of additional surgeries to correct defects, break down scar tissue (capsulotomy or capsulaectomy), to replace the implant, or remove the implant.

Implant surfaces have been made both smooth and textured in an attempt to reduce encapsulation, however, neither has been proven to produce consistently superior results. Animal models suggest that there is an increased tendency for increased capsular thickness and contracture with textured surfaces that encourage fibrous tissue ingrowth on the surface. In addition, an implanted medical device can trigger a “foreign body” response where the immune system recognizes the implanted medical device as foreign and triggers acytokine reaction that ultimately leads to scar tissue formation. A specific form of foreign body reaction in response to medical device placement is a complete enclosure (“walling off”) of the implanted medical device in a capsule of scar tissue (encapsulation). Fibrous encapsulation of the implanted medical devices compromises or impairs the function of the medical device.

Carbon monoxide (CO) is, by common definition, a colorless, odorless, tasteless, non-corrosive gas of about the same density as that of air and is the most commonly encountered and pervasive poison in our environment. It is generally produced by the incomplete combustion of fossil fuels such as natural gas, propane, coal, gasoline and wood. In the atmosphere, the average global levels are estimated to be 0.19 parts per million (ppm), 90% of which comes from natural sources including ocean micro-organism production, and 10% of which is generated by human activity Thus, inhalation of even small quantities of CO is inevitable for living organisms.

Depending on the extent and time of exposure, CO is capable of producing a myriad of debilitating and harmful residual effects to the organism. The most immediate of these effects, and perhaps the most notorious one, is binding to hemoglobin in the blood stream, which rapidly decreases the oxygen transport capability of the cardiovascular system. Paradoxically, more than half a century ago it was found that CO is constantly formed in humans in small quantities, and that under certain pathophysiological conditions this endogenous production of CO may be considerably increased. The discovery that hemoglobin, a heme-dependent protein, is required as substrate for the production of CO in vivo and the identification of the enzyme heme oxygenase as the crucial pathway for the generation of this gaseous molecule in mammals set the basis for the early investigation of an unexpected and still unrecognized role of CO in the vasculature. The succeeding cloning and characterization of constitutive (HO-2) and inducible (HO-1) isoforms of heme oxygenase as well as studies on the kinetics and tissue distribution of these enzymes started to reveal a major importance of this pathway in the physiological degradation of heme. That is, the end products of heme degradation (CO, biliverdin and bilirubin) might possess, after all, crucial biological activities.

With regard to the cardiovascular system, the recognition that CO possesses vasodilatory properties is, perhaps, the most significant evidence in favor of a pharmacological function of CO. Although the molecular mechanisms and the chemical modifications that are required to transduce the signals mediated by CO into a specific biological effect need to be fully elucidated, convincing scientific reports have documented the signaling properties of endogenously generated CO. Additionally, recent studies have also shown the importance of the roles of CO in the immune, respiratory, reproductive, gastrointestinal, kidney, and liver systems. The understanding of the cellular and molecular mechanisms that regulate the production and mediate the physiological actions of CO has greatly advanced in recent years. Many diseases, including neurodegenerations, hypertension, heart failure, and inflammation, have been linked to the abnormality in CO metabolism and function.

As detailed in U.S. Patent Application 2002/0155166 by Choi et al., novel pharmaceutical compositions for delivering to patients suffering from the effects of oxidative stress are disclosed, the compositions comprising effective concentrations of carbon monoxide in a gaseous mixture comprising oxygen and optionally, nitrogen gas (as well as other minor optional gaseous components). Choi et al. disclose the unexpected discovery that the delivery of a therapeutic gas comprising low concentrations (i.e., concentrations ranging from about 1 ppb (part per billion) to about 3,000 ppm (such as above about 0.1 ppm within this range) of the gas, such as about 1 ppm to about 2,800 ppm, such as about 25 ppm to about 750 ppm, such as about 50 ppm to about 500 ppm) of carbon monoxide is an extremely effective method for delaying the onset of, inhibiting or reversing the effects of oxidative stress in a patient. Still another aspect of the invention disclosed by Choi et al. relates to a method for inhibiting the production of proliferative cytokines such as TNF-α, IL-1β, IL-6, MIP-1β and augmenting the production (expression) of the anti-proliferative cytokines IL-10 and IL-4 in a patient comprising administering to the patient an effective amount of gaseous CO. A problem with the use of gaseous CO as a pharmaceutical composition is the need for specialized equipment to administer the gaseous pharmaceutical composition. This specialized equipment includes gas cylinders, valves, etc.

U.S. Pat. No. 7,045,140 discloses metal carbonyl (e.g., ruthenium-based carbonyls) compounds and methods for delivering said metal carbonyls to induce vasodilation, attenuate vasoconstriction, modulate blood pressure, and delay transplant rejection. This invention demonstrates that systemic delivery of metal carbonyls can exert biological and physiological effects. A problem with systemic delivery of CO is that it binds to hemoglobin with ˜200× higher affinity than oxygen, which may lead to formation of carboxyhemoglobin (COHb). Thus, the systemic system serves as a sink to absorb circulating CO. As COHb levels increase, less hemoglobin is available for the transport of oxygen. Prolonged exposure to systemic administration of CO may lead to carbon monoxide poisoning and/or death. Additionally, a problem with ruthenium-based compounds is that they are poisonous, highly toxic, and carcinogenic.

U.S. Patent Application 2006/0148900 discloses CO-containing organic, inorganic, and organometallic complexes, and CO-containing substances, that release CO either by an enzymatic process or by decarbonylation. Although this invention discloses the preparation of nine CO-containing compounds, the release of CO has not been demonstrated. Further, it is not clear if any of these compounds will have a biological effect. Lastly, it is not clear how the compounds will be used, in addition to how the compounds will be administered.

U.S. Pat. No. 5,882,674 proposes administration of CO via transdermal delivery systems containing metal carbonyl complexes such as iron pentacarbonyl and iron enneacarbonyl. However, since this document provides no experimental data, and no description of specific systems, it is not clear how this proposal can be made to work. Also, this transdermal system contemplates that the active substance reaches systemic circulation. The CO-related problems therewith are described above.

U.S. Patent Applications 2004/0052866, 2004/0228930, 2004/0131703, 2004/0052866, 2004/0005367, 2003/0219497, and 2003/0039638 demonstrate CO treatment methods utilizing liquid or gaseous compositions. The gaseous or liquid form can be administered to the patient by any method known in the art for administering gases and liquids to patients, e.g., via inhalation, insufflation, infusion, injection, and/or ingestion. One skill in the art can readily appreciate that these applications contemplate the systemic delivery of CO. Systemic administration of such drugs in sufficient amounts to supply an efficacious concentration to the local treatment site often produces adverse or toxic side effects for the patient. A further problem with the use of gaseous CO as a pharmaceutical composition is the inability to incorporate into materials.

U.S. Patent Application 2003/0219496 teaches the use of CO for treating intimal hyperplasia, a disease of smooth muscle cell over-proliferation, as a result of using a balloon angioplasty device. In one embodiment, this application teaches that exposing animals to gaseous CO for one hour at 250 ppm prior to a balloon injury can prevent intimal hyperplasia two weeks later. In another embodiment, the application contemplates a balloon angioplasty device and atherectomy device designed to deliver CO while angioplasty and atherectomy is being performed, respectively. The application does not demonstrate a use of such devices, so it is unclear if such devices will actually provide effects. Additionally, it can be readily appreciated by those in the art that these devices are to be used in a short time frame, and are not meant to be used long-term.

Additionally, the 2003/0219496 application teaches a stent, coated with a CO-releasing coating, in order to treat intimal hyperplasia of the blood vessel. Although the application contemplates that the stent is a long-term medical device, an actual working stent has not been demonstrated so it is unclear if such a stent would actually provide an effect on blood vessel intimal hyperplasia, or smooth muscle cell over-proliferation.

Fibrotic encapsulation of medical devices is an important problem. There has been little success in developing compositions for use in medical devices that can minimize the formation of fibrotic encapsulation.

The approaches for using CO described in the prior art for stents was to treat intimal hyperplasia of the blood vessel and not to prevent fibrosis and/or fibrotic encapsulation of medical devices. Furthermore, the approaches described for stents are less than practical to prevent fibrosis and/or fibrotic encapsulation of medical devices.

Until now, the art failed to disclose the use of CO to prevent fibrosis. Furthermore, the art did not disclose the use of CO to prevent fibrosis and/or fibrotic encapsulation of medical devices. Still furthermore, the art did not disclose the use of CO incorporated into materials to prevent fibrosis and/or fibrotic encapsulation of medical devices that are implant for long periods of time. Thus, what remains is the need for a fibrosis-inhibiting composition delivered locally from the soft tissue implant or implanted medical device, or administered locally into the tissue surrounding the soft tissue implant or medical device, which can minimize fibrous tissue formation, encapsulation and capsular contracture resulting therefrom.

### SUMMARY OF THE INVENTION

The present invention relates to anti-fibrotic compositions comprising non-gaseous CO dissolved in a reservoir material (“CO Materials”), wherein said composition is used to minimize fibrotic encapsulation resulting from a soft tissue implant and/or medical device. Upon exposure to a biological environment, the non-gaseous CO is slowly desorbed or diffused from the reservoir material to provide anti-fibrotic activity. These compositions may be used directly on tissue to treat and/or prevent conditions and disease states related to fibrosis. Furthermore, these compositions can be incorporated into materials that are used to create soft tissue implants and medical devices to provide said implants and devices with anti-fibrotic properties. Further still, these compositions can be incorporated into materials that are used to coat soft tissue implants and medical devices to provide said implants and devices with anti-fibrotic properties.

Reservoir materials useful in this invention that are able to provide a reservoir for regulated release of carbon monoxide include liquid compositions. The liquid material can be mixed homogenously with the polymer material. Furthermore, the liquid material can be encapsulated by the polymer material. The controlled rate of release allows localized CO delivery for extended periods, e.g., seconds, minutes, hours, weeks to months, depending upon the application.

These liquid compositions can be used to create a soft tissue implant or a medical device for regulated release of carbon monoxide to provide anti-fibrotic activity. These liquid compositions can be used to coat a soft tissue implant or a medical device for regulated release of carbon monoxide to provide anti-fibrotic activity. Additionally, these liquid compositions can be incorporated into a polymer composition, wherein the liquid material acts as the reservoir for the CO and the polymer material allows for a controlled rate of release of CO from a reservoir material.

Further, reservoir materials useful in this invention include polymer compositions that are able to provide a reservoir for regulated release of carbon monoxide. These polymer compositions can consist of single polymer material that acts as a CO reservoir and provides for regulated release of carbon monoxide. Further, these polymer compositions can consist of two or more polymer materials wherein the first polymer material acts as the reservoir for the CO and the second polymer material allows for a controlled rate of release of CO from a polymer composition. The controlled rate of release allows localized CO delivery for extended periods, e.g., seconds, minutes, hours, weeks to months, depending upon the application. This is especially useful in providing therapy to reduce or prevent fibroblast proliferation in a localized area.

The anti-fibrotic compositions using reservoir materials in the polymer form can be used to create soft tissue implants and medical devices to provide said implants and devices with anti-fibrotic properties. The anti-fibrotic compositions using reservoir materials in the polymer form can be used to coat soft tissue implants and medical devices to provide said implants and devices with anti-fibrotic properties.

Still further, reservoir materials useful in this invention include liquid and/or polymer compositions that include agents that enhance the amount of CO in the reservoir materials. These CO-enhancing agents aid the reservoir material in absorbing CO. Particular CO-enhancing agents include materials that contain one metal or a mixture of metals selected from Ni, Mn, Rh, Cu, and Ag. The metals maybe be comprised in a zeolite support. The metal complex may comprise a metal ion and a metal ion ligand, such as penicillamine, glutathione, porphyrin, chlorophyll, hemoglobin, or ETDA. The reservoir material may comprise a polymer composition, such as gas-permeable polymers, non gas-permeable polymers, or mixtures thereof, silicone, or polyurethane. The composition, when not physically-associated with a device or implant, may be provided topically, orally, parenterally, subcutaneously, intravenously, intramuscularly, or intraperitoneally.

The reservoir material may be comprised in a medical device, such as gastrointestinal stents, tracheal/bronchial stents, genital-urinary stents, ENT stents, intra-articular implants, intraocular lenses, implants for hypertrophic scars and keloids, vascular grafts, anastomotic connector devices, implantable sensors, implantable pumps, implantable electrical devices, such as implantable neurostimulators and implantable electrical leads, surgical adhesion barriers, glaucoma drainage devices, surgical films and meshes, prosthetic heart valves, tympanostomy tubes, penile implants, endotracheal and tracheostomy tubes, peritoneal dialysis catheters, intracranial pressure monitors, vena cava filters, central venous catheters (CVCs), ventricular assist devices (e.g., LVAD), spinal prostheses, urinary (Foley) catheters, prosthetic bladder sphincters, orthopedic implants, and gastrointestinal drainage tubes. The reservoir material may also be comprised in a soft tissue implant, such as saline breast implants, silicone breast implants, triglyceride-filled breast implants, hyaluronic acid-based implants, chin and mandibular implants, nasal implants, cheek implants, lip implants, and other facial implants, pectoral and chest implants, malar and submalar implants, and buttocks implants.

Also provided is method for providing anti-fibrotic activity, comprising administering to a patient in need of such an effective amount of a composition comprising non-gaseous CO and a reservoir material, wherein fibrotic activity results from a soft tissue implant and/or medical device, wherein the materials and devices are as described above.

Another embodiment provides a kit comprising a composition of non-gaseous CO and a reservoir material, wherein said composition is used to prevent fibrosis resulting from a soft tissue implant and/or medical device, wherein the materials and devices are as described above.

A particular embodiment comprises a breast implant medical device that is less prone to fibrotic encapsulation comprising a polymer shell and a filler, wherein the filler has dissolved within it carbon monoxide and said carbon monoxide desorbs from the filler and diffuses through the polymer shell to provide antifibrotic and anti-inflammatory properties to said breast implant medical device. The breast implant medical device may comprises saline or silicone gel.

Also provided specifically is a method of providing to a patient breast implant medical devices that are less prone to fibrotic encapsulation comprising at least (a) inserting the polymer shell into the patient; and (b) filling the polymer shell with a filler material, wherein filler material as has dissolved within it carbon monoxide.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 and FIG. 2 illustrate that silicone and copper chlorophyllin silicone can act as reservoirs for CO delivery.

FIG. 3 demonstrates that polymeric material may be used to regulate CO release.

FIG. 4 illustrates that silicone CO and saline silicone CO can mitigate cell proliferation of dermal fibroblasts.

FIG. 5 and FIG. 6 are graphs depicting that silicone CO and saline silicone CO can attenuate TNFα and IL-1β release from macrophages.

FIG. 7 and FIG. 8 illustrate the CO gas released from a saline filled, CO-saturated breast implant and from CO gas-filled breast implants.

### DETAILED DESCRIPTION OF THE INVENTION

The present invention relates to anti-fibrotic compositions comprising non-gaseous CO dissolved in a reservoir material, wherein said composition is used to prevent fibrotic encapsulation resulting from a soft tissue implant and/or medical device. Upon exposure to a biological environment, the non-gaseous CO is slowly desorbed and/or diffused from the reservoir material to provide anti-fibrotic activity. These compositions can be incorporated into materials that are used to create soft tissue implants and medical devices to provide said implants and devices with anti-fibrotic properties. Furthermore, these compositions can be incorporated into materials that are used to coat soft tissue implants and medical devices to provide said implants and devices with anti-fibrotic properties.

### I. LIQUID RESERVOIR MATERIALS

The anti-fibrotic compositions using reservoir materials in the liquid form can be used to create a soft tissue implant and/or a medical device to provide said implant and/or device with anti-fibrotic properties. These liquid compositions can be used to coat a soft tissue implant or a medical device to provide said implant and/or device with anti-fibrotic properties.

In one embodiment, reservoir materials useful in this invention that are able to provide a reservoir for regulated release of carbon monoxide include liquid compositions such as water and saline, and liquid polymers such as silicone, polyethylene glycol (PEG), triglyceride and silicone gel.

In another embodiment, liquid materials can comprise a medical device and/or soft tissue implant, such as glycosaminoglycans (e.g., hyaluronic acid). Hyaluronic acid is a soft tissue implant that is used in subdermal or dermal facial tissue injections to smooth out wrinkles. Carbon monoxide dissolved in glycosaminoglycans can provide an antifibrotic effect to mitigate fibrosis.

In one embodiment, these liquid compositions can be incorporated into a polymer composition wherein the liquid material acts as the reservoir for the CO and the polymer material allows for a controlled rate of release of CO from a reservoir material. The liquid material can be mixed homogenously with the polymer material. Furthermore, the liquid material can be encapsulated by the polymer material. The controlled rate of release allows localized CO delivery for extended periods, e.g., seconds, minutes, hours, weeks to months, depending upon the application.

### II. POLYMER RESERVOIR MATERIALS

The anti-fibrotic compositions using reservoir materials in the polymer form can be used to create a soft tissue implant and/or a medical device to provide said implant and/or device with anti-fibrotic properties. The anti-fibrotic compositions using reservoir materials in the polymer form can be used to coat a soft tissue implant and/or a medical device to provide said implant and/or device with anti-fibrotic properties.

In one embodiment, reservoir materials useful in this invention include polymer compositions that are able to provide a reservoir for regulated release of carbon monoxide. These polymer compositions can consist of a single polymer material that acts as a CO reservoir and provides for regulated release of carbon monoxide.

In one embodiment, these polymer compositions can consist of two or more polymer materials wherein the first polymer material acts as the reservoir for the CO and the second polymer material allows for a controlled rate of release of CO from a polymer composition. The controlled rate of release allows localized CO delivery for extended periods, e.g., seconds, minutes, hours, weeks to months, depending upon the application. This is especially useful in providing therapy to reduce or prevent fibroblast proliferation in a localized area.

In one embodiment, the polymeric material may be comprised of a gas-permeable polymer. In another embodiment, the polymeric material may be comprised of a combination of a gas-permeable and a non gas-permeable polymer to regulate the release of CO. Gas-permeable and non gas-permeable polymers are known in the art, and may be hydrophilic or hydrophobic. Without limitation, examples of gas-permeable and non gas-permeable polymers are polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, polyolefins, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Polymer dispersions such as polyurethane dispersions and acrylic latex dispersions are also within the scope of the present concept.

The gas-permeable and non gas-permeable polymer matrix may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example.

In one embodiment, the polymer may be useful in coating or creating medical devices, such as gastrointestinal stents, tracheal/bronchial stents, genital-urinary stents, ENT stents, intra-articular implants, intraocular lenses, implants for hypertrophic scars and keloids, vascular grafts, anastomotic connector devices, implantable sensors, implantable pumps, implantable electrical devices, such as implantable neurostimulators and implantable electrical leads, surgical adhesion barriers, glaucoma drainage devices, surgical films and meshes, prosthetic heart valves, tympanostomy tubes, penile implants, endotracheal and tracheostomy tubes, peritoneal dialysis catheters, intracranial pressure monitors, vena cava filters, central venous catheters (CVCs), ventricular assist devices (e.g., LVAD), spinal prostheses, urinary (Foley) catheters, prosthetic bladder sphincters, orthopedic implants, and gastrointestinal drainage tubes.

In another embodiment, the polymer may be useful in coating or creating soft tissue implants such as saline breast implants, silicone breast implants, triglyceride-filled breast implants, hyaluronic acid-based implants, chin and mandibular implants, nasal implants, cheek implants, lip implants, and other facial implants, pectoral and chest implants, malar and submalar implants, and buttocks implants.

One skilled in the art can readily appreciate that variations in the polymer matrix composition may be utilized to achieve the desired regulation and control of CO delivery. For example, long-term catheters may be better suited for a prolonged release of CO, whereas short-term catheters may be better suited for shorter release periods of CO. Designing the CO release characteristics of the polymeric matrix around the intent of the medical device (i.e. short-term, intermediate-term, long-term) are envisioned to lower side effects of the medical device, such as fibrosis and fibrotic encapsulation.

### III. CO-ENHANCING AGENTS

A further aspect of CO-enhancing agents of this invention is the use of reservoir materials that include agents that enhance the amount of CO in the reservoir materials. These CO-enhancing agents aid the reservoir material in absorbing CO.

In one embodiment, the CO-enhancing agent consists of reservoir materials that include water-insoluble CO-enhancing particles. As used within this disclosure, water-insoluble CO-enhancing particles refer to particles that do not dissolve or only slowly dissolve in the presence of water.

These insoluble CO-enhancing particles can be incorporated in a pharmaceutical or cosmetic base for topical delivery of CO. Likewise, the insoluble CO-enhancing particles can be incorporated into a material (such as polymer, ceramic, textile, etc.) to provide the surface of said material with anti-fibrotic properties.

In the simplest form, the water-insoluble CO-enhancing agent can be a CO absorptive material such as activated charcoal, activated alumina, silica (e.g., fumed, gel, etc.). In this form, the carbon monoxide is simple adsorbed into the material. When placed in an appropriate environment, the carbon monoxide slowly desorbs providing therapeutic benefit.

In a particular embodiment, the water insoluble CO-enhancing agent contains one metal or a mixture of metals selected from Ni, Mn, Rh, Cu, and Ag. The metals can be adsorbed into CO absorptive materials such as activated charcoal, activated alumina, silica (e.g., fumed, gel, etc.). However, in a particular embodiment, the water-insoluble CO-enhancing agent consists of a zeolite containing one or more metals.

The use of zeolite absorptive materials has been disclosed in the prior art for separation of carbon monoxide in gas mixtures. Please see U.S. Pat. Nos. 4,019,879, 4,587,114, 4,717,398, and 4,743,276 for examples of zeolite absorptive materials useful for this concept. In one embodiment, the zeolite absorptive material is a zeolite carrier selected from the group consisting of Y-type zeolite and mordenite-type zeolite and having a silica/alumina ratio of not more than 10, an effective pore diameter of not less than 0.38 nm (3.8 A).

Examples of a zeolite carrier may include Y-type zeolite (Na₂O Al₂O₃4 8SiO₂. 8 9H₂O), A-type zeolite (Na₂O Al₂O₃2SiO₂ 4.5H₂O), mordenite (Na₂O Al₂O Al₂O₃ 9-10SiO₂ 6H₂O), X-type zeolite (Na₂O Al₂O₃2 5SiO₂ 6H₂O), L-type clinoptilolite, Ω-type clinoptilolite, erionite, faujasite, ZK-4, ZSM-2, ZSM-3, ZSM-4 and ZSM-10. A zeolite carrier can be a natural or synthetic zeolite (U.S. Pat. No. 4,743,276).

For a CO-enhancing agent, one particular embodiment uses zeolite as a CO-enhancing agent in the reservoir with a high CO/N₂ adsorption ratio (vCO/vN₂). For example, NiY, MnY and RhY have high CO/N₂ adsorption ratios (vCO/vN₂) of about 2.5 or more, and Cu(I)—Y, AgY and AgX have extremely high CO/N₂ adsorption ratios of about 6 or more. The reason for this is surmised to be as follows. When a selected carrier contains Ni, Mn or Rh, the carried cations cause a change in pore diameter of the zeolite to allow easier adsorption of CO than N₂. Cu(I) and Ag cations which are transition group elements have a valency of 1 and have a good affinity for CO. A synergetic effect of these two effects is also plausible (U.S. Pat. No. 4,743,276).

One example of CO-enhancing agent for use in the reservoir of the CO-enhancing agent is a silver ion containing zeolite such as Ag—Y. Another example of a silver zeolite material is disclosed in U.S. Pat. No. 4,019,880. In this patent, crystalline zeolitic molecular sieves were those species that have pore diameters large enough to adsorb CO and which have or are modified to have framework SiO₂/Al₂O₃ molar ratios of from 20 to 200, more specifically from 20 to 100. A number of synthetic zeolite species are available which have sufficiently high SiO₂/Al₂O₃ molar ratios in the as-synthesized form. These include zeolite Ω, zeolites ZSM-5, ZSM-8, ZSM-11 and ZSM-12 as disclosed in detail in U.S. Pat. Nos. 3,702,886 and 3,640,681. The zeolite having SiO₂/Al₂O₃ molar ratios increased by such means to the range of 20 to 200 are satisfactory for the present process.

The silver cation forms of the zeolite adsorbents utilized in the present process are readily prepared by conventional ion-exchange techniques using aqueous solutions of silver salts such as silver nitrate. One advantage of using silver as the CO absorptive metal is that the silver ions provide antimicrobial properties in addition to the anti-fibrotic properties provided by the CO.

Another form of a CO-enhancing agent useful for incorporation in the reservoir of the CO-enhancing agent includes “water-soluble glasses.” Water-soluble glasses are well-known in the art and are described as way of example in U.S. Pat. Nos. 5,330,770, 5,290,544, and 5,470,585. Typically, water-soluble glasses are made of one or two glass-forming oxides also known as glass formers, e.g., silicon dioxide, boric oxide, and phosphorus pentoxide in combination with one or more of glass modifiers such as calcium oxide, sodium oxide, potassium oxide, zinc oxide, barium oxide, magnesium oxide, and mixtures thereof. For example, aforesaid U.S. Pat. No. 5,330,770, describes a boron-free water-soluble glass manufactured from a silicon dioxide as the glass former, and sodium oxide as the glass modifier. Silver oxide is incorporated into the water-soluble glass as an antibacterial agent. Aforesaid U.S. Pat. No. 5,290,544, describes a water-soluble glass including silicon dioxide or phosphorus pentoxide as the glass former and calcium oxide, potassium oxide or sodium oxide as the glass modifier. The glass further includes at least one metal ion such as Ag⁺, Cu⁺ and Zn²⁺ having an antibacterial property. Water-soluble glasses are also commercially available, e.g., ARGLAES® glass (Giltech Limited, Great Britain).

### IV. WATER-SOLUBLE CO-ENHANCING AGENTS

In another embodiment, the CO-enhancing agents are water-soluble. The water soluble CO-enhancing materials can be dissolved or mixed in with the liquid reservoir material and administered to a patient to treat and/or prevent fibrotic conditions and disease states.

The water-soluble CO-enhancing agents of the present invention may, in particular, contain one metal or a mixture of metals selected from Ni, Mn, Rh, Cu, and Ag. A specific metal is Cu(I) or Ag. Other metals useful in this concept include platinum, palladium, rhenium, iridium, and the carbides and nitrides of tungsten, molybdenum, vanadium, chromium, tantalum and mixtures thereof. Upon exposure to a biological environment, the adsorbed CO is slowly desorbed from the carrier material providing therapeutic effects.

The water-soluble CO-enhancing agents can be dissolved directly into the liquid reservoir materials that make up the CO-enhancing agents and administered to a patient to treat conditions and disease states. Likewise, the water-soluble CO-enhancing agents can be incorporated into another material so as to provide said material anti-fibrotic properties.

Examples of water-soluble CO-enhancing agents useful for incorporation into the reservoir of the CO-enhancing agents are water-soluble compounds that are able to bind to specific metal ions selected from Ni, Mn, Rh, Cu, and Ag. These include chelators, porphirin ring type compounds, etc. Examples of water-soluble CO-enhancing agent include penicillamine, glutathione, ETDA, porphyrin ring type materials, chlorophyll materials, etc.

Specific examples of water-soluble CO-enhancing agents useful in this concept include penicillamine-Cu(I), glutathione-Cu(I), copper (II) phthlocyanine, silver thiosulfate complex, coppered chlorophyllin (trisodium salt), hemoglobin, protophorhirin, etc.

### V. OTHER ENHANCING AGENTS

One skilled in the art can readily appreciate that other agents can be added to the CO materials to enhance the antifibrotic properties. For example, anti-inflammatory therapeutics such as steroids can be included in the CO material to provide additional benefits. As way of another example, halofuginone, which is known to inhibit collagen deposition in fibrous capsules, can be added to the CO material to provide added benefit. This invention contemplates the combination of other active agents in the CO material that can aid in the prevention of fibrosis.

### VI. MAKING AND USING CO MATERIALS

The process for making CO materials is accomplished as follows. First, the reservoir material is selected. The selection of the reservoir material for the CO material depends on the purpose for using the CO material. If the purpose of the CO material is to prevent or treat fibroblast proliferation at the site of a medical device, then the reservoir material can be a polymer. The polymer reservoir material can be the same material that comprise the polymer device (i.e., silicone rubber in a catheter) or can be polymer coating (i.e., polymer-coated infusion pump). If the end-use is a medical implant such as a breast implant consisting of a silicone polymer shell filled with liquid silicone or saline, then the liquid filler for the silicone implant can be selected as the reservoir material.

Second, the CO material's reservoir material is loaded with CO. Again, depending on the end-use will dictate how the reservoir material is treated. In the case of a polymer medical device (i.e., silicone rubber catheter) wherein the polymer material is the reservoir material, the medical device is treated in a chamber containing CO. If the reservoir material is a liquid, the CO is bubbled through the reservoir material.

Third, the CO material is packaged. After the CO material in the end-product (i.e., device, solution, etc.) has been loaded with CO, the end-product needs to packaged in a manner to minimize the adsorption of the CO from the end-product. In the case of a polymer catheter product, after the product as been made, packaged, sterilized and loaded with CO, the product is then packaged in a container that is not permeable to CO. For example, the finished catheter product is sealed in a foil-lined packaged. For products containing liquid reservoir materials, the process is the same. For example, in cases of the liquid filler for breast implants, the liquid that is loaded with CO is provided to the doctor in a CO impermeable container. When the doctor is filling the breast implant, he or she would transfer the liquid CO material from the container to the implant using standard means.

When not used as part of the device or implant, the administration of the composition may be by almost any route, including topically, orally, parenterally, subcutaneously, intravenously, intramuscularly, intraperitoneally, etc. It may also be provided locally or regionally with respect to an implant, device or implant site.

A. Making CO Material Utilizing Water-Soluble CO-Enhancing Agents

The basic process to making a CO material utilizing water-soluble CO-enhancing agents useful for this disclosure involves the following steps:

1. Produce or acquire a CO-enhancing agents. For example, an excellent CO-enhancing agent is a metal-containing zeolite. Please refer to U.S. Pat. Nos. 4,019,879, 4,587,114, 4,717,398, and 4,743,276 for examples of producing metal containing zeolite absorptive materials that would be useful as CO-enhancing agent useful for this invention. Additionally, silver-exchange zeolite may be purchased through chemical supply companies known in the art, such as Aldrich (see Aldrich Chemical 38, 228-0). In a manner similar to producing silver-exchange zeolite, copper-exchange zeolite and other monovalent ion-exchange zeolite could be made. The water-soluble CO-enhancing agents containing a metal can be acquired or produced using standard techniques well know in the art. For silver thiosulfate production, please see U.S. Pat. No. 6,093,414. For penicillamine-Cu(I) and glutathione-Cu(I) see references 2 and 3. Another example of a water-soluble carrier material is copper chloropyllin trisodium salt (See Aldrich 25, 828-8).

2. Mix the CO-enhancing agent with the liquid or solid reservoir material. In the case of a water-insoluble CO-enhancing agent, the CO-enhancing agent can be mixed into the reservoir material. For example, the reservoir material is a polymer, the water-insoluble CO-enhancing agent can be mixed into the polymer material prior to curing the polymer material. After curing, the solidified reservoir material contains the CO-enhancing agent directly incorporated into the material. The CO-enhancing agent aids in holding CO in the reservoir material. In the case of a water-soluble CO-enhancing agent, the CO-enhancing agent can be mixed or dissolved into the reservoir material. For example, if the reservoir material is saline, the water-soluble CO-enhancing agent can be dissolved into the reservoir material. The CO-enhancing agent aids in holding CO in the liquid reservoir material.

3. Load CO into the reservoir material. In general, CO can be applied under pressure, elevated temperature or other means to have it be adsorbed by the reservoir material. Typically, CO should be provided in a pure form so as to drive the absorption into the CO material. Please refer to U.S. Pat. Nos. 4,019,879, 4,587,114, 4,717,398, and 4,743,276, and Examples 6-10, for description of absorption CO to materials.

4. CO material is packaged. After the CO material in the end-product (i.e., device, solution, etc.) has been loaded with CO, the end-product needs to packaged in a manner to minimize the adsorption of the CO from the end-product.

B. Therapeutic Uses of CO Material

The CO material of the present invention may be incorporated into materials that are used to create or coat soft tissue implants and/or medical devices to prevent fibrosis associated with said implants and/or devices.

The term “fibrosis” as used herein is used to describe the formation or development of excess fibrous connective tissue in an organ or tissue that is associated with a reparative or reactive process.

By way of illustration, CO material may be incorporated in a polymer that is used to create or coat a central line catheter. The CO material provides the surface of the central line catheter with anti-fibrotic properties thus minimizing the initiation of cytokine release and fibroblast proliferation. By way of another illustration, the CO material may be incorporated into the filler for breast implants to provide anti-fibrotic properties to the implant to minimize fibrosis encapsulation.

By way of an addition illustration, the CO material may be used to create breast implants that are less prone to fibrotic encapsulation. In one embodiment, the breast implant consists of a silicone shell that is filled with CO material consisting of CO dissolved in saline. In another embodiment, the breast implant consists of a silicone shell that is filled with CO material consisting of CO dissolved in silicone gel. In both embodiments, the doctor places the silicone shell in the patient unfilled and then fills the shell with the CO material. The silicone shell acts to regulate the release of the CO from the CO material. CO desorbs from the CO material into the surrounding tissue preventing the formation of a fibrotic capsule.

Similarly, the CO material, in the form of water-soluble (and water-insoluble) CO material, may be used in cosmetics and personal care products to provide these products with anti-fibrotic properties. Examples of cosmetics include lipsticks and glosses, lip pencils, mascara, eye liners, eye shadows, moisturizers, liquid and powder makeup foundations, powder and cream blushes, perfumes, colognes, various creams and toners, etc., and assorted applicators like combs, brushes, sponges, and cotton swabs and balls, and examples of personal care products include deodorants, razors, shaving creams, shampoos, conditioners, various hair treatments like mousses and sprays, toothpastes, mouthwashes, dental flosses and tapes, sunscreens, moisturizers, tampons, sanitary napkins, panty shields, diapers, baby wipes, facial tissues, toilet tissues, etc.

Whereas particular aspects of the present concept and particular embodiments of the invention have been described hereinbefore for purposes of illustration, it will be appreciated by those skilled in the art that numerous variations of the details may be made without departing from the concept as described in the appended claims.

### VII. KITS

Any of the compositions described herein may be comprised in a kit. In one embodiment, the compositions are comprised in a kit comprising a composition of non-gaseous CO and a reservoir material, wherein said composition is used to prevent fibrosis resulting from a soft tissue implant and/or medical device. The non-gaseous CO may comprise carbon monoxide adsorbed to a carrier material, wherein the carrier material contains one metal or a mixture of metals selected from group of Ni, Mn, Rh, Cu, and Ag. In a further embodiment, the carrier material comprises a zeolite containing one metal or a mixture of metals selected from Ni, Mn, Rh, Cu, and Ag. Alternatively, the carrier material comprises a metal complex containing one metal or a mixture of metals selected from Ni, Mn, Rh, Cu, and Ag. The metal complex may comprise a metal ion and a metal ion ligand. In some embodiments, the metal ion ligand is pencillamine, glutathione, porphyrin, chlorophyll, hemoglobin, ETDA, etc. The reservoir material may be a polymer composition. In some embodiments, the polymer composition may comprise gas-permeable polymers, non gas-permeable polymers, or mixtures thereof. Alternatively, the polymer composition may comprise silicone, polyurethane, etc. In some embodiments, the composition may be provided topically, orally, parenterally, subcutaneously, intravenously, intramuscularly, intraperitoneally, etc.

In some embodiments, the reservoir material is comprised in a medical device. The medical device may be gastrointestinal stents, tracheal/bronchial stents, genital-urinary stents, ENT stents, intra-articular implants, intraocular lenses, implants for hypertrophic scars and keloids, vascular grafts, anastomotic connector devices, implantable sensors, implantable pumps, implantable electrical devices, such as implantable neurostimulators and implantable electrical leads, surgical adhesion barriers, glaucoma drainage devices, surgical films and meshes, prosthetic heart valves, tympanostomy tubes, penile implants, endotracheal and tracheostomy tubes, peritoneal dialysis catheters, intracranial pressure monitors, vena cava filters, central venous catheters (CVCs), ventricular assist devices (e.g., LVAD), spinal prostheses, urinary (Foley) catheters, prosthetic bladder sphincters, orthopedic implants, and gastrointestinal drainage tubes. In a particular embodiment, the reservoir material is comprised in a soft tissue implant. The soft tissue implant may be saline breast implants, silicone breast implants, triglyceride-filled breast implants, hyaluronic acid-based implants, chin and mandibular implants, nasal implants, cheek implants, lip implants, and other facial implants, pectoral and chest implants, malar and submalar implants, and buttocks implants.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional containers into which the additional components may be separately placed. However, various combinations of components may be comprised in a vial. The kits of the present invention also will typically include a means for containing the component containers in close confinement for commercial sale. Such containers may include cardboard or injection or blow-molded plastic containers into which the desired vials, bottles, etc. are retained.

When the components of the kit are provided in one or more liquid solutions, the liquid solution may be an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

A kit may also include instructions for employing the kit components. Instructions may include variations that can be implemented.

### VIII. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute specific modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### CO Material of a Solid Polymer Reservoir

It can be readily appreciated by those in the art that the type of polymer utilized can be selected based on the desired regulation of CO release and the medical use of the polymer. In one example, medical grade silicone was used. The polymer was injected into a cylindrical mold. It can be readily appreciated by those in the art that the type of mold used can be dependent of the medical use. As an alternative, the polymer can coat a medical device. The polymer sample was allowed to cure within the cylindrical mold for an appropriate amount of time. After the polymer was cured, the polymer sample was placed in a gas chamber and exposed to CO gas for CO adsorption for 72 hours. It can be readily appreciated by those in the art that the polymer sample may be placed in the gas camber for a longer period of time depending of the amount of polymer material and type of polymer material utilized.

### Example 2

### CO Material of a Liquid Reservoir Encapsulated within a Polymer

It can be readily appreciated by those in the art that the type of polymer utilized can be selected based on the desired regulation of CO release and the medical use of the polymer. Additionally, it can be readily appreciated by those in the art that the type of CO-enhancing agent utilized can be selected based on the desired regulation of CO release and the medical use of the CO-enhancing agent. Additionally, it can be readily appreciated by those in the art that the type of liquid utilized can be selected based on the desired medical use of the liquid. In one example, medical grade silicone, copper chlorophyllin, and saline solution were used. The CO-enhancing agent was admixed with a polymer material. Five hundred milligrams of Copper chlorophyllin was admixed with 9.5 g medical grade silicone to create a 10% CO-enhancing agent embedded within the silicone. It can be readily appreciated by those in the art that the amount of CO-enhancing agent embedded in the silicone can be less than 1% to greater than 50%. The 10% copper chlorophyllin in silicone was injected into a cylindrical mold leaving a lumen to be filled with a saline solution. It can be readily appreciated by those in the art that the type of mold used can be dependent of the medical use. As an alternative, the CO-enhancing agent in polymer can coat a medical device. The 10% copper chlorophyllin in silicone was allowed to cure within the cylindrical mold for an appropriate amount of time. The saline solution was exposed to CO gas in a gas chamber to dissolve to CO gas within the saline solution. After the 10% copper chlorophyllin in silicone was cured, the lumen was filled with saline solution. The 10% copper chlorophyllin in silicone, encapsulating the saline solution, was placed in a gas chamber and exposed to CO gas for CO adsorption for 72 hours. It can be readily appreciated by those in the art that the CO-enhancing agent/polymer, encapsulating the saline solution, may be placed in the gas chamber for a longer period of time depending of the percentage and amount of CO-enhancing agent embedded within the polymer material and type of polymer material utilized.

### Example 3

### CO Material of a Polymer Reservoir Incorporating a CO-Enhancing Agent

It can be readily appreciated by those in the art that the type of polymer utilized can be selected based on the desired regulation of CO release and the medical use of the polymer. Additionally, it can be readily appreciated by those in the art that the type of CO-enhancing agent utilized can be selected based on the desired regulation of CO release and the medical use of the CO-enhancing agent. In one example, medical grade silicone and copper chlorophyllin were used. The CO-enhancing agent was admixed with a polymer material. Five hundred milligrams of copper chlorophyllin was admixed with 9.5 g medical grade silicone to create a 10% CO-enhancing agent embedded within the silicone. It can be readily appreciated by those in the art that the amount of CO-enhancing agent embedded in the silicone can be less than 1% to greater than 50%. The 10% copper chlorophyllin in silicone was injected into a cylindrical mold. It can be readily appreciated by those in the art that the type of mold used can be dependent of the medical use. As an alternative, the CO-enhancing agent in polymer can coat a medical device. The 10% copper chlorophyllin in silicone was allowed to cure within the cylindrical mold for an appropriate amount of time. After the 10% copper chlorophyllin in silicone was cured, the 10% copper chlorophyllin in silicone was placed in a gas chamber and exposed to CO gas for CO adsorption for 72 hours. It can be readily appreciated by those in the art that the CO-enhancing agent/polymer may be placed in the gas chamber for a longer period of time depending of the percentage and amount of CO-enhancing agent embedded within the polymer material and type of polymer material utilized.

### Example 4

### Release of CO by CO Material

Carrier gas was produced from compressed air that was scrubbed through a Hopkalite catalytic converter (custom-made) operated at 100° C. at a flow rate of 30 mL/min. The resulting CO-“free” air contained <2 ppb CO. Levels of CO were determined by a custom built solid-phase gas chromatography unit (Peak Laboratories) that is sensitive to 0.1 ppb, exquisitely selective for CO and is unaffected by nitrogen. The CO peak was digitally recorded and concentration determined by quantitation of CO peak area. The analyzer was calibrated daily before experiments with 0 to 1000 ppb CO standards. The sensitivity of CO measurements was reliably >2 ppb.

The CO-releasing reactions were carried out in a 15 mL polyethylene chamber fitted with gas tight tubing leading to the GC unit. The chamber was vented with CO-free air prior to each experiment. For determination of CO generation, the CO-releasing sample or control was sealed in the chamber which was continuously vented with CO free carrier gas (30 mL/min). At 2 minute intervals, the headspace outflow was diverted into the GC analyzer for determination of CO concentration. Rate of CO generation was calculated as the product of the chamber flow net outflow concentration of CO. For ease of interpretation, final CO levels were translated from ppb to molar using 24.5 L/mole gas (1 atm and 25° C.) as a conversion factor.

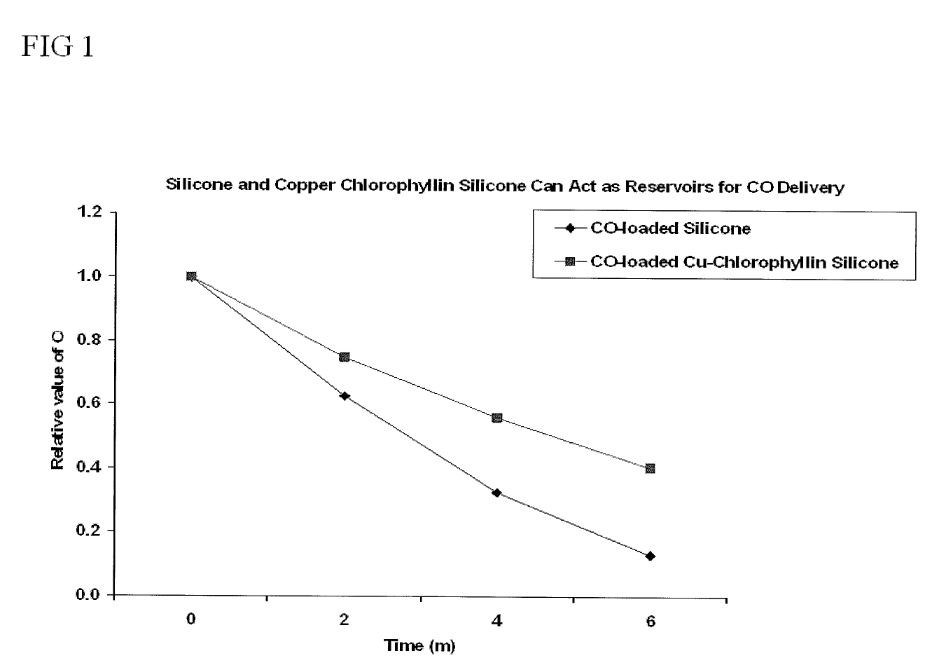

Silicone (Example 1) and copper chlorophyllin silicone (Example 3) material was made. As way of illustration, 500 milligrams of copper chlorophyllin (Example 3) was blended with 9.5 grams of silicone rubber. As by way of another illustration, 10 grams of silicone was not blended with any CO-enhancing agent (Example 1). Both silicone rubber samples were allowed to cure. From these cured silicone samples, 80-100 milligrams smaller samples were created for placement in CO gas for adsorption. The 80-100 milligram samples were placed in polyethylene chamber as described above for measurement of CO release by the sample. As demonstrated in FIG. 1, the CO slowly dissociated from both the CO-containing copper chlorophyllin silicon and CO-containing silicone, with the CO-enhancing agent embedded silicone demonstrating higher release rate of CO.

### EXAMPLE 5

### Regulated Release of CO by a Polymeric Matrix

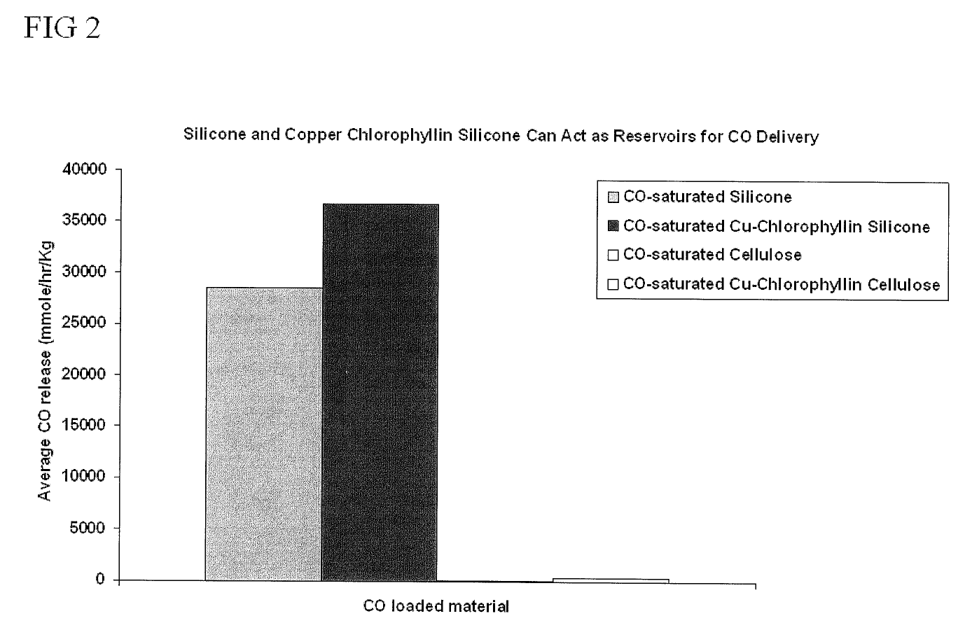

Silicone (Example 1) and copper chlorophyllin in silicone (Example 3) was made. As way of example, 9.5 grams of silicone rubber was blended with 500 milligrams of copper chlorophyllin (Example 3) or without any copper chlorophyllin (Example 1). Both silicone rubber samples were allowed to cure. From these cured silicone samples, 80-100 milligrams smaller samples were created for placement in CO gas for adsorption. As by way of another example, 10 milligrams of cellulose was blended with 1 milligram of copper chlorophyllin or without copper chlorophyllin. The samples were placed in polyethylene chamber as described above for measurement of CO release by the sample. As demonstrated in FIG. 2, the CO slowly dissociated from both CO-containing samples, with the CO-enhancing agent embedded silicone demonstrating higher average release rate of CO. Additionally, although the lighter weight of the cellulose polymer has been corrected for, the cellulose polymer is unable to provide any appreciable release of CO. Additionally, the copper chlorophyllin blended with the cellulose provided some release of CO, indicating that the CO-enhancing agent can provide for a reservoir of CO release.

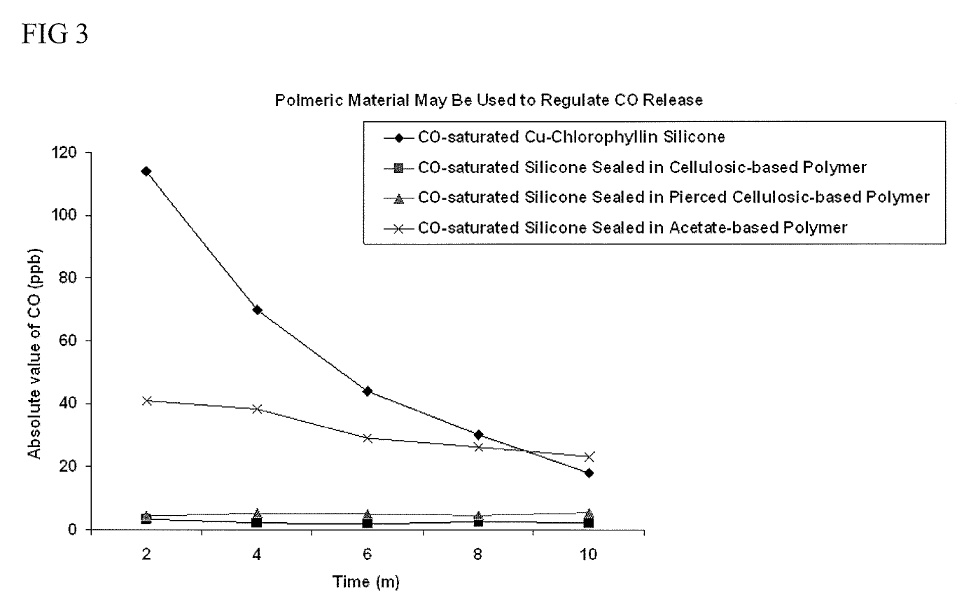

In order to further explore the result above, that is the inability of cellulose to provide for release of CO, additional polymer materials were explored. Silicone and copper chlorophyllin embedded silicone were prepared as described above. Following CO gas adsorption, the samples were sealed in different polymeric material (see FIG. 3). The samples were placed in polyethylene chamber as described above for measurement of CO release by the sample. As demonstrated in FIG. 3, silicone sealed in cellulose polymer did not release CO, but pierced cellulose did release a small amount of CO. Additionally, silicone sealed in an acetate-based polymer demonstrated a controlled and regulated release of CO. The copper chlorophyllin silicone released CO much more quickly.

### Example 6

### Anti-Fibrotic Effects of Polymeric CO Material

Human dermal fibroblasts were seeded into culture flasks and grown at 37±2° C. and 5±1% CO₂ using Fibroblast Growth Medium (FGM). When a sufficient number of cells grew they were resuspended in DMEM+1.5% FBS, seeded into to a 24-well plate (reserving one empty well for non-specific binding) and cultured overnight to allow the cells to adhere to the well plates. On the following day the culture media was replaced with FGM in all wells except the positive control (reduced proliferation) well, which will continue to receive DMEM+1.5% FBS. The test materials were loaded into 6.5 mm transwell inserts and the transwell inserts were then inserted into the wells of the 24 well plate. The inserts containing the material brought the test material to within 1 mm of the fibroblast monolayer at the bottom of the well. Enough media was added to each well to ensure that the test material within the transwell was also submerged.

After one day of incubation 80 μl of BrdU solution was added to the wells and the plates were incubated overnight. On the next day the culture media was removed via aspiration and 800 μl of a fixative/DNA denaturing agent was added and the plate was allowed to incubate for approximately 30 minutes at room temperature. After removing the fixative solution via aspiration, 400 μl of an anti-BrdU antibody solution was added to each well and the plate was incubated for 1 hour at room temperature. After washing the plate 3× with wash buffer, 400 μl of the peroxidase conjugated secondary antibody solution was added to each well and the plate was incubated for approximately 30 minutes at room temperature. After washing the plate 3× with wash buffer and once with distilled water, 400 μl of the substrate solution was added to each well and the plate was incubated for 15 minutes at room temperature in the dark. After the 15-minute incubation, 400 μl of stop solution was added to each well and the well plate was read at 460 nm with a correction for the well plate at 540 nm.

Absorbance values made at 460 nm was corrected by subtracting their corresponding 540 nm absorbance values (to correct for variation in the plastic of the 24-well plate). After correcting for both variation in the well plate and nonspecific background, the mean 460 nm absorbance was calculated for each treatment and used to represent the amount of DNA synthesis.

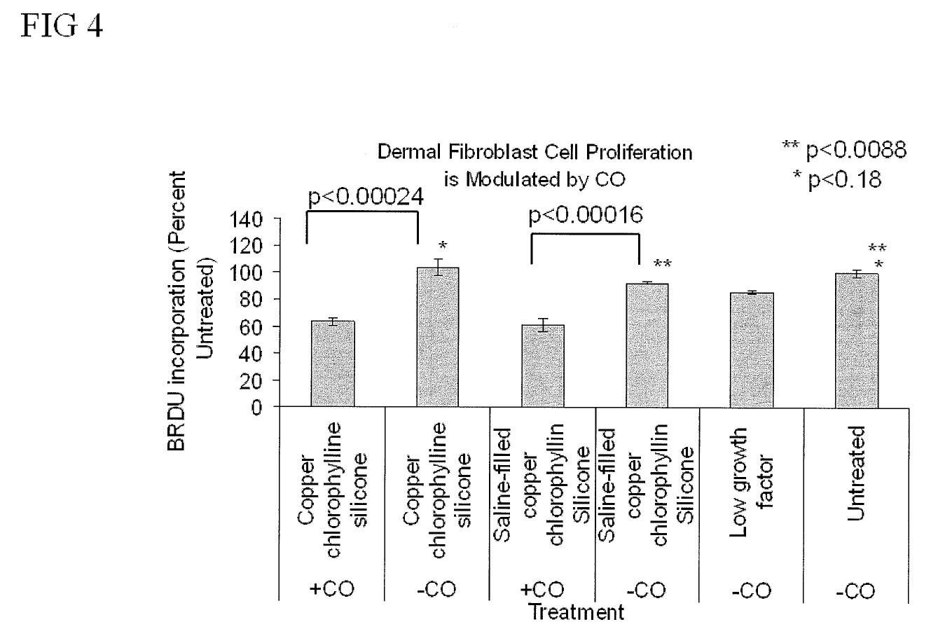

An anti-fibrotic material was made by incorporating the CO-enhancing agent of Example 3 into a silicone polymer. However, the copper chlorophyllin embedded silicone polymer was extruded into a longer cylindrical mold for cell culture testing. In addition, another set of samples was made by filling the lumen of the cylindrical mold with CO dissolved in saline (CO-saline-silicone) or saline without CO, and encapsulating the saline solution with copper chlorophyllin embedded silicone (Example 2). When the copper chlorophyllin embedded silicone samples were placed in the transwell insert in cell culture, the CO slowly dissociated from the CO Material and polymeric matrix to provide reduced (˜40%) proliferation (p<0.00024) (See FIG. 4). When the CO-saline-silicone samples were placed in the transwell insert in cell culture, the CO slowly dissociated from the CO Material and polymeric matrix to provide reduced (˜30%) proliferation (p<0.00016) (See FIG. 4). The control silicone sample did not have a statistical effect (p<0.18).

### Example 7

### Effects of Cytokine Release from Polymeric CO Material

Cytokine response in the RAW macrophages was initiated by adding 10 ng/mL LPS. For a negative control samples, RAW macrophages were exposed to the LPS stimuli but will not be treated with any type of anti-fibrotic material. One additional set of samples was left without exposure to the LPS stimuli to provide a baseline measurement for the cytokines. The macrophages were incubated at 37±2° C. and 5±1% CO₂ for the duration of the experiment. After the experiment, the cell culture medium was collected and stored at −75° C. until analyzed for cytokines.

IL-1β and TNFα were detected using ELISA kits specific for each cytokine. A series of standards were prepared for each cytokine. An ELISA microplate was prepared by removing any unneeded strips from the plate frame. 100 μl of each sample or standard was dispensed into two wells, while two empty wells were reserved for use as blanks later in the assay. 100 μl of acetylcholinesterase:anti-TNFα FAB conjugate solution or acetylcholinesterase:anti-IL-1β FAB conjugate solution, depending upon the ELISA, was added to each well used (with the exception of the blanks) and the plate was covered and incubated overnight at 2-8° C. overnight. After the overnight incubation the wells was rinsed 3-5 times with wash buffer. After the final wash was removed, 200 μl of Ellman's Reagent was added to each well and the plate was developed at room temperature protected from light. Absorbance measurements were made at 405 nm using a plate reader.

To quantify the amount of TNFα or IL-1β present, a standard curve was generated using known concentrations of each cytokine. A linear regression was performed to establish the line that best fits these data points. Mean absorbance values for the test materials and untreated samples were used to estimate the amount of each cytokine present in each sample.

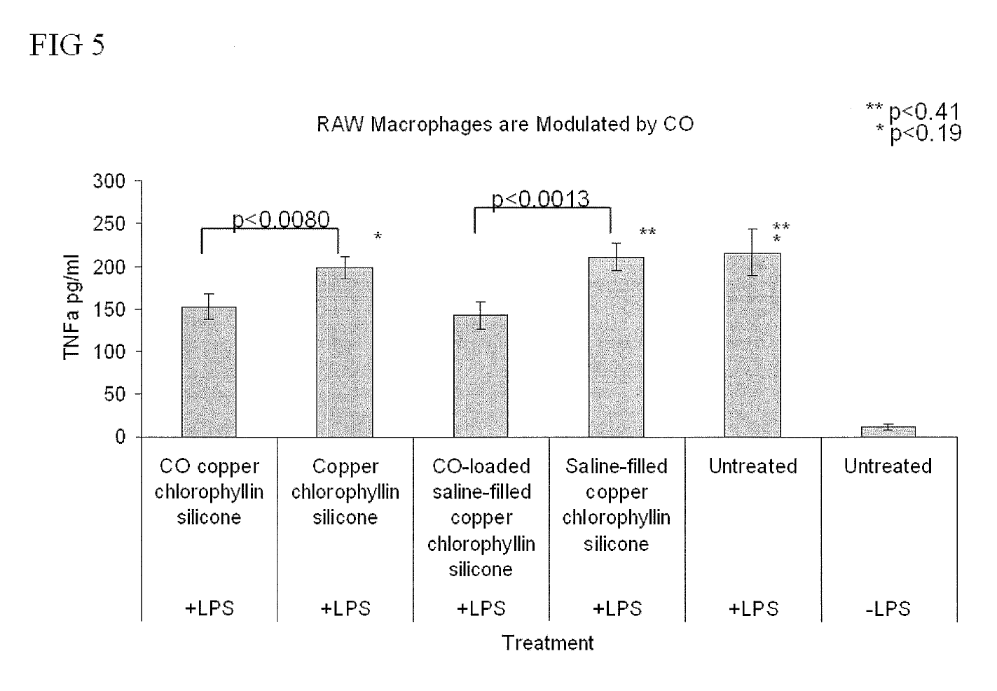

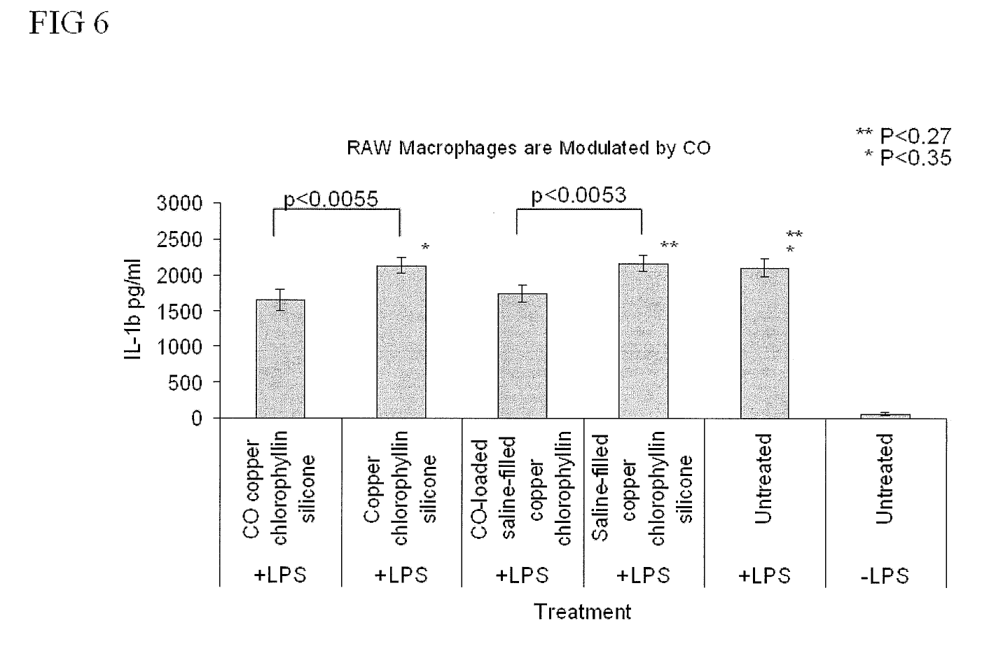

An anti-fibrotic material was made by incorporating the CO-enhancing agent of Examples 1 and 3 into a silicone polymer in the same fashion as Example 6 above. However, the copper chlorophyllin silicone polymer was extruded into a longer cylindrical mold for cell culture testing. In addition, another set of samples was made by filling the lumen of the cylindrical mold with CO dissolved in saline (CO-saline-silicone) or saline without CO, and encapsulating the saline solution with copper chlorophyllin embedded silicone (Example 2). When the copper chlorophyllin embedded silicone samples were placed in the transwell insert in cell culture, the CO slowly dissociated from the CO Material and polymeric matrix to provide reduced (˜25% for TNFα; ˜33% for IL-1β) cytokine release (p<0.0080; p<0.0055) (see FIG. 5 and FIG. 6). When the CO-saline-silicone samples were placed in the transwell insert in cell culture, the CO slowly dissociated from the CO Material and polymeric matrix to provide reduced (˜33% for TNFα; ˜23% for IL-1β) cytokine release (p<0.0013; p<0.0053) (see FIG. 5 and FIG. 6). The control samples without CO did not have a statistical effect (p<0.19 and p<0.41 for TNFα; p<0.35 and p<0.27 for IL-1β).

### Example 8

### Regulated Release of CO by a Medical Device

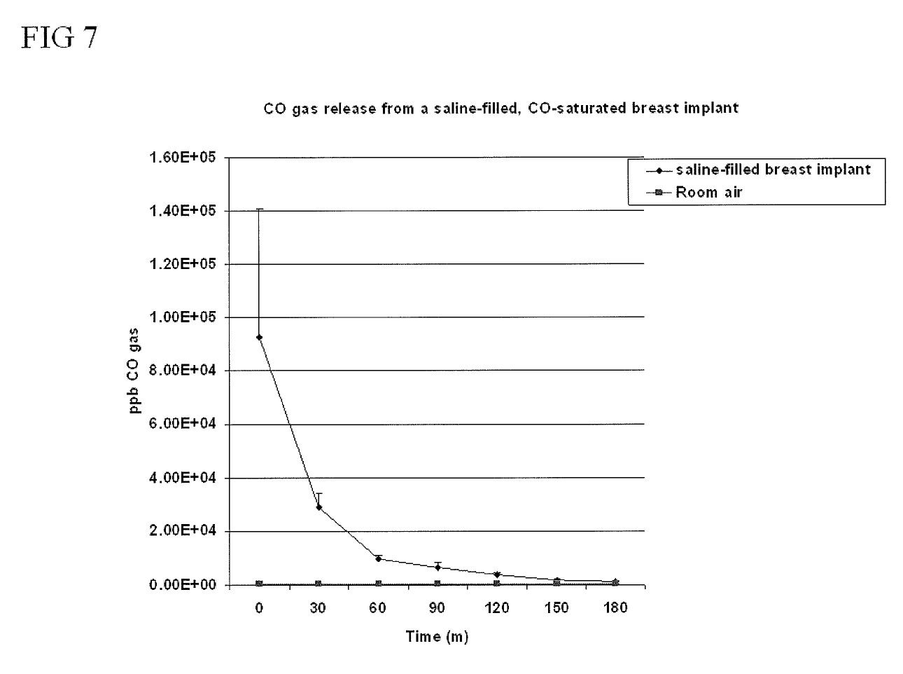

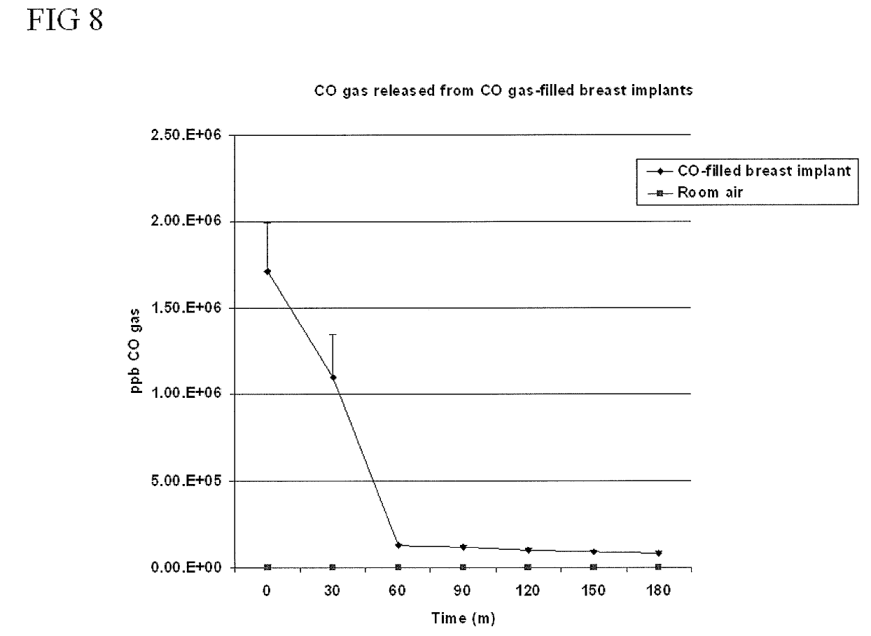

Twenty milliliter breast implants were obtained as a gift from a major breast implant manufacturer. One set of breast implants was filled with pure CO air. Another set of breast implants was filled with saline and exposed to pure CO air, and flushed several times to reach saturation of the breast implant with CO. Each breast implant was placed in glass bottles for 5 minutes, at which time a sample of air was removed from the glass bottle for measurement of CO as described above to calculate the release of CO by the breast implant. As demonstrated in FIG. 7 and FIG. 8, the CO slowly dissociated from both CO-filled breast implants and saline-filled, CO-saturated, breast implant. The breast implants containing a CO enhancing agent, embedded within the breast implant shell and/or provided with the saline solution, would demonstrate a higher average release rate of CO, and would indicate that the CO enhancing agent can provide for a reservoir of CO release.

The slow release rate of CO from the breast implant reservoir, once implanted in a mammal, would reduce the amount of inflammation and fibroblast growth around the implant, thus reducing capsular contraction.

All of the compositions and methods disclosed and claimed herein can be made and executed without undue experimentation in light of the present disclosure. While the compositions and methods of this invention have been described in terms of exemplified embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference:
U.S. Pat. No. 4,019,879
U.S. Pat. No. 4,019,880
U.S. Pat. No. 4,587,114
U.S. Pat. No. 4,717,398
U.S. Pat. No. 4,743,276
U.S. Pat. No. 5,882,674
U.S. Pat. No. 5,909,736
U.S. Pat. No. 6,093,414
U.S. Pat. No. 7,045,140
U.S. Publn. 20020155166
U.S. Publn. 2004/0005367
U.S. Publn. 2004/0052866
U.S. Publn. 2004/0131703
U.S. Publn. 2004/0228930
U.S. Publn. 2004/0258772
U.S. Publn. 2006/0148900
Coburn et al., J. Clin. Invest., 42:1172-1178, 1963.
Coburn et al., J. Clin. Invest., 45:460-468, 1966.
Coburn et al., J. Clin. Invest., 46:346-356, 1967.
Coceani et al., Am. J. Physiol., 246(4 Pt 2):H640-3, 1984.
Coceani, Circ. Res., 86(12):1184-6, 2000.
Furchgott et al., Blood Vessels, 28:52-61, 1991.
Kato et al., J. Inorg. Biochem., 75(2):117-21, 1999.
Maines et al., J. Biol. Chem., 261:411-9, 1986.
Maines, FASEB J., 2:2557-68, 1988.
Marks et al., Trends Pharmacol. Sci., 12:185-8, 1991.
McDonagh, Clin. Perinat., 17:359-69, 1990.
Morita et al., Proc. Natl. Acad. Sci. USA, 92:1475-9, 1995.
Osterberg et al., Inorg. Biochem., 10(4):341-55, 1979.
Piantadosi, In: Carbon monoxide, Penney (Ed.), Chapter 8, 1996.
Scharf, J. Appl. Physiol., 39:752-8, 1975.
Shibahara et al., Proc. Natl. Acad. Sci. USA, 82:7865-9, 1985.
Sjostrand, Scan. J. Clin. Lab. Invest., 1:201-14, 1949.
Sjostrand, Acta Physiol. Scand., 26:328-33, 1952.
Tenhunen et al., J. Biol. Chem., 244:6388-94, 1969.
Trakshel et al., J. Biol. Chem., 264:1323-8, 1989.
Zhou et al. Am. J. Pathol., 166(1):27-37, 2005.

1. A composition comprising non-gaseous CO dissolved in a reservoir material, wherein said non-gaseous CO desorbs and/or diffuses from the reservoir material, wherein said composition is used to minimize fibrotic proliferation resulting from a soft tissue implant and/or medical device.

2. The composition of claim 1, wherein the non-gaseous CO comprises carbon monoxide adsorbed to a carrier material wherein the carrier material contains one metal or a mixture of metals selected from group of Ni, Mn, Rh, Cu, and Ag.

3. The composition of claim 2, wherein the carrier material comprises a zeolite comprising one metal or a mixture of metals selected from Ni, Mn, Rh, Cu, and Ag.

4. The composition of claim 2, wherein the carrier material comprises a metal complex comprising one metal or a mixture of metals selected from Ni, Mn, Rh, Cu, and Ag.

5. The composition of claim 4, wherein the metal complex comprises a metal ion and a metal ion ligand.

6. The composition of claim 5, wherein the metal ion ligand is selected from the group consisting of pencillamine, glutathione, porphyrin, chlorophyll, hemoglobin, and ETDA.

7. The composition of claim 1, wherein the reservoir material comprises a polymer composition.

8. The composition of claim 7, wherein the polymer composition comprises gas-permeable polymers, non gas-permeable polymers, or mixtures thereof.

9. The composition of claim 7, wherein the polymer composition comprises silicone and polyurethane.

10. The composition of claim 7, wherein the polymer composition further comprises a carrier material, wherein the carrier material contains one metal or a mixture of metals selected from Ni, Mn, Rh, Cu, and Ag.

11. The composition of claim 10, wherein the carrier material comprises a zeolite containing one metal or a mixture of metals selected from Ni, Mn, Rh, Cu, and Ag.

12. The composition of claim 10, wherein the carrier material comprises a metal complex containing one metal or a mixture of metals selected from Ni, Mn, Rh, Cu, and Ag.

13. The composition of claim 12, wherein the metal complex comprises a metal ion and a metal ion ligand.

14. The composition of claim 13, wherein the metal ion ligand is selected from the group consisting of pencillamine, glutathione, porphyrin, chlorophyll, hemoglobin, and ETDA.

15. The composition of claim 1, wherein the reservoir material is comprised in a medical device.

16. The composition of claim 15, wherein the medical device is selected from the group consisting of gastrointestinal stents, tracheal/bronchial stents, genital-urinary stents, ENT stents, intra-articular implants, intraocular lenses, implants for hypertrophic scars and keloids, vascular grafts, anastomotic connector devices, implantable sensors, implantable pumps, implantable electrical devices, such as implantable neurostimulators and implantable electrical leads, surgical adhesion barriers, glaucoma drainage devices, surgical films and meshes, prosthetic heart valves, tympanostomy tubes, penile implants, endotracheal and tracheostomy tubes, peritoneal dialysis catheters, intracranial pressure monitors, vena cava filters, central venous catheters (CVCs), ventricular assist devices (e.g., LVAD), spinal prostheses, urinary (Foley) catheters, prosthetic bladder sphincters, orthopedic implants, and gastrointestinal drainage tubes.

17. The composition of claim 1, wherein the reservoir material is comprised in a soft tissue implant.

18. The composition of claim 17, wherein the soft tissue implant is selected from the group consisting of saline breast implants, silicone breast implants, triglyceride-filled breast implants, hyaluronic acid-based implants, chin and mandibular implants, nasal implants, cheek implants, lip implants, and other facial implants, pectoral and chest implants, malar and submalar implants, and buttocks implants.

19. The composition of claim 15, wherein the implant or device comprises a carrier material, wherein the carrier material contains one metal or a mixture of metals selected from Ni, Mn, Rh, Cu, and Ag.

20. The composition of claim 19, wherein the carrier material comprises a zeolite containing one metal or a mixture of metals selected from Ni, Mn, Rh, Cu, and Ag.

21. The composition of claim 19, wherein the carrier material comprises a metal complex containing one metal or a mixture of metals selected from Ni, Mn, Rh, Cu, and Ag.

22. The composition of claim 21, wherein the metal complex comprises a metal ion and a metal ion ligand.

23. The composition of claim 22, wherein the metal ion ligand is selected from the group consisting of pencillamine, glutathione, porphyrin, chlorophyll, hemoglobin, and ETDA.

24. (canceled)

25. A method for providing anti-fibrotic activity comprising administering to a patient in need of such an effective amount of a composition comprising non-gaseous CO and a reservoir material, wherein fibrotic activity results from a soft tissue implant and/or medical device.

26-38. (canceled)

39. The method of claim 25, wherein the reservoir material is comprised in a medical device.

40. The method of claim 39, wherein the medical device is selected from the group consisting of gastrointestinal stents, tracheal/bronchial stents, genital-urinary stents, ENT stents, intra-articular implants, intraocular lenses, implants for hypertrophic scars and keloids, vascular grafts, anastomotic connector devices, implantable sensors, implantable pumps, implantable electrical devices, such as implantable neurostimulators and implantable electrical leads, surgical adhesion barriers, glaucoma drainage devices, surgical films and meshes, prosthetic heart valves, tympanostomy tubes, penile implants, endotracheal and tracheostomy tubes, peritoneal dialysis catheters, intracranial pressure monitors, vena cava filters, central venous catheters (CVCs), ventricular assist devices (e.g., LVAD), spinal prostheses, urinary (Foley) catheters, prosthetic bladder sphincters, orthopedic implants, and gastrointestinal drainage tubes.

41-45. (canceled)

46. The method of claim 25, wherein the reservoir material is comprised in a soft tissue implant.

47. The method of claim 46, wherein the soft tissue implant is selected from the group consisting of saline breast implants, silicone breast implants, triglyceride-filled breast implants, hyaluronic acid-based implants, chin and mandibular implants, nasal implants, cheek implants, lip implants, and other facial implants, pectoral and chest implants, malar and submalar implants, and buttocks implants.

48-52. (canceled)

53. The method of claim 25, wherein the composition is provided topically, orally, parenterally, subcutaneously, intravenously, intramuscularly, or intraperitoneally.

54. A kit comprising a composition of non-gaseous CO and a reservoir material, wherein said composition is used to prevent fibrosis resulting from a soft tissue implant and/or medical device.

55-82. (canceled)

83. A breast implant medical device that is less prone to fibrotic encapsulation comprising a polymer shell and a filler, wherein the filler has dissolved within it carbon monoxide and said carbon monoxide desorbs from the filler and diffuses through the polymer shell to provide antifibrotic and anti-inflammatory properties to said breast implant medical device.

84. The breast implant medical device of claim 83, wherein the filler comprises saline or silicone gel.

85. A method of providing to a patient breast implant medical devices that are less prone to fibrotic encapsulation comprising:
(a) inserting the polymer shell into the patient; and
(b) filling the polymer shell with a filler material, wherein filler material as has dissolved within it carbon monoxide.
